(19) United States
(12) Patent Application Publication
(10) Pub. No.: US 2003/0175297 A1
(43) Pub. Date: Sep. 18, 2003

(54) Cancer vaccines

(76) Inventor: Mitsuyoshi Urashima, Tokyo (JP)

Correspondence Address: CLARK & ELBING LLP, 101 FEDERAL STREET, BOSTON, MA, 02110, US

(21) Appl. No.: 10/346,940

(22) Filed: Jan. 17, 2003

Related U.S. Application Data
(63) Continuation of application No. 09/524,315, filed on Mar. 13, 2000, now abandoned.

(30) Foreign Application Priority Data
Jun. 29, 1999 (JP) 11/183376

(51) Int. Cl. A61K 39/02; C12N 1/21

(52) U.S. Cl. 424/200.1; 435/252.3

(57) ABSTRACT

The present invention provides cancer vaccines that highly induce tumor immunity through oral administration or other administration routes, uses thereof, and the production methods thereof. The cancer vaccine is the cells of a microorganism that can transfer exogenous genes into the host cell, wherein the cells expressibly maintain DNA capable of inducing tumor immunity in the host, and can induce tumor immunity by themselves.

### FIELD OF THE INVENTION

The present invention relates to vaccine preparations that exhibit antitumor activities through such administration routes as oral administration.

### BACKGROUND OF THE INVENTION

Recently genetic immunization has been in practice, in which nonpathogenic bacteria are transformed with a DNA expression vector containing a target gene and inoculated into the body of an animal through oral administration and other routes. Darji et al. demonstrated the efficient vaccination against Listeria monocytogenesis in mice by the use of an oral somatic transgene by using attenuated Salmonella into which a plasmid carrying the Listeria toxin gene is introduced (A. Darji et al., Cell 91: 765-775, 1997). The orally inoculated Salmonella bacterium reaches the Peyer's patch by penetrating through digestive tract epithelial M cells, and is taken up by macrophage and others through phagocytosis (B. D. Johns et al., J. Exp. Med. 180: 15-23, 1994). The Peyer's patch is a specialized lymphatic tissue which is distributed in the jejunum and the ileum and contains the M cells that carry antigens captured in the intestines to lymphoid tissues (Mennekigaku Jiten (Dictionary of Immunology); Tokyo Kagaku Dojin). A plasmid in the Salmonella bacterium is released into the cytosol and integrated into the nucleus, where its gene expression is induced under the control of an eukaryotic promoter. In the meantime the Salmonella bacterium is known to die out after several rounds of cell division because it cannot proliferate due to its aroA mutation (aromatic amino acids synthesis deficiency). Thus the attenuated Salmonella bacterium is expected to be useful as a gene carrier which transports the expression vector to the host, targeting the digestive tract tissues.

What kinds of genes besides the Listeria toxin gene can be introduced by the attenuated Salmonella bacterium has been confirmed for the tetanus toxin gene (U.S. Pat. No. 5,547,664). In this case, the expression of the introduced gene successfully produced the therapeutic effect. The attenuated Salmonella bacterium has also been utilized for the development of vaccines against microorganisms for which the usual vaccines were not sufficiently effective (such as pertussis toxin and Helicobacter pylori). Moreover, some papers have recently suggested its effectiveness against an autoimmune disease (SLE) (M. L. Huggins et al., Lupus 8: 29-38, 1999; P. T. Dalla et al., Vaccine 16: 22-29, 1998; P. K. Fagan et al., Infect. Immune. 65: 2502-2507, 1997; and O. G. Gomez-Duarte et al., Vaccine 26: 1667-1673, 1999).

Although others have been reported on their attempts to treat tumors by intraperitoneal administration of the attenuated Salmonella bacterium, it has been pointed out that such an administration method may risk the host (Cancer Res. 57:4537-4544, 1997; and Nature Biotech. 17:37-41, 1999).

CD40 ligand (CD40L) is expressed on the surface of activated CD4⁺ T cells, basophils, and mast cells. Binding of CD40L to its receptor, CD40, on the surface of B cells stimulates B cell proliferation, differentiation, and immunoglobulin (Ig) class switching (E. A. Clark and J. A. Ledbetter, Nature 367: 425-428, 1994). Moreover, CD40L is also required to activate antigen presenting cells (APCs), macrophages, and dendritic cells (DCs). This result in an upregulation in the expression of CD80 (B7-1), and CD86 (B7-2) on the APCs, this in turn, engages the CD28 receptor on T cells, resulting in a reciprocal amplification of antigen specific T cells as well as cytotoxic T cells and natural killer cells (S. R. Bennett et al., Nature 393: 478-480, 1998; P. Borrow et al., J. Exp. Med. 183: 2129-2142, 1996; I. S. Grewal and J. Xu, Nature 378: 617-620, 1995; and E. Carbone et al., J. Exp. Med. 185: 2053-2060, 1997). CD40 is also expressed on the surface of malignant cells derived from the B cell lineage. CD40 ligation upregulated expression of not only basophil molecules, MHC class I/II, and co-stimulatory molecules (B7 family), but also the Ku antigen and possibly tumor antigens on malignant B cells, which may trigger tumor-specific immunity (F. M. Uckun et al., Blood 76: 2449, 1990; M. Urashima et al., Blood 8:1903-1912, 1995; and G. Teoh et al., J. Clin. Invest. 101: 1379-1388, 1998). Indeed, CD40L has a growth suppressive effect on malignant B cells both in vivo and in vitro in contrast to the effects of CD40L on normal immune cells (A. A. Cardoso et al., Blood 90: 549-561, 1997). Therefore, immune-genetherapy utilizing CD40L may be a potential treatment for the B cell lymphoma (BCL).

As described above, the induction of the CD40L expression in the host would be useful for cancer immunotherapy that takes advantage of the tumor immunity induction of CD40L. As one of the methods to induce the CD40L expression in the host, one can think of a method, for example, in which the DNA encoding CD40L is expressibly introduced into the host and is forcedly expressed. A technology called DNA vaccine is usually employed to introduce an exogenous gene into the host cells. However, DNA vaccine is an advanced medical technology which must meet high levels of both safety and efficacy requirements. Therefore, safer and more effective DNA vaccine that can induce the CD40L expression in the host cells has been awaited.

### SUMMARY OF THE INVENTION

An objective of the present invention is to provide a novel DNA vaccine that can effectively induce tumor immunity by introducing an exogenous gene into the host cells.

The present inventors have thought that new cancer immunotherapy will be realized by more safely introducing and effectively expressing the DNA encoding a protein, such as CD40L, that can induce tumor immunity in the host. The present inventors orally administered the attenuated Salmonella bacterium transformed with a vector that can express CD40L to mice, and observed the effect of tumor immunity induction. As a result, the use of the attenuated Salmonella bacterium not only enabled the introduction and expression of the exogenous gene, CD40L, but also induced a high level of tumor immunity in the host, partially helped by the antitumor effects of the attenuated Salmonella bacterium itself. Based on these findings, the present inventors have completed the invention.

The present invention relates to cancer vaccines, uses thereof, and production methods thereof shown below.

(1) A cancer vaccine comprising a microbial cell that can expressibly maintain a DNA capable of inducing tumor immunity in a host and transfer the DNA into a host cell, wherein the microbial cell induce tumor immunity by itself.

(2) The cancer vaccine of (1), wherein the microbial cell is attenuated Salmonella bacterium.

(3) The cancer vaccine of (2), wherein the attenuated Salmonella bacterium is an auxotrophic aroA⁻ strain of Salmonella typhimurium.

(4) The cancer vaccine of (2) or (3), wherein the vaccine is a preparation for oral administration.

(5) The cancer vaccine of (1), wherein the DNA encodes a CD40 ligand.

(6) A method of preventing of the recurrence of hematopoietic cancer, the method comprising administering the cancer vaccine of (5).

a) A method of producing a cancer vaccine, the method comprising the steps of:

a) constructing a tumor immunity induction vector comprising a DNA encoding a polypeptide that induces tumor immunity in a host when the vector is introduced into the host cell;

b) transforming a microbial cell, which can transfer an exogenous gene into a host cell and induce the tumor immunity by itself, with the tumor immunity induction vector; and

collecting the transformants to formulate them into a cancer vaccine for oral administration.

(7) A potentiator of the tumor immunity induction effect of a CD40 ligand comprising attenuated Salmonella bacterium.

### BRIEF DESCRIPTION OF THE DRAWINGS

FIG. 1 is graphs showing the results of flow cytometry examining the effects of human CD40L on the expression of Fas, B7-1, and B7-2. The top, middle, and bottom panels show the expression of Fas, B7-1, and B7-2, respectively. The ordinate represents the number of cells, and the abscissa the intensity of fluorescence. The three graphs on the right-hand side are the results with CD40L, and the three on the left-hand those with the vector alone as controls.

FIG. 2 is a graph showing the temporal changes in concentrations of the soluble human CD40L secreted into the serum of the mouse into which the DNA encoding CD40L has been introduced using the cancer vaccine of the present invention. The ordinate represents the concentrations of the soluble CD40L, and the abscissa the time passed (in weeks).

FIG. 3 is a graph showing protection against tumor challenge with ST40L, comparing among the groups inoculated with ST40L, with ST alone, and with PBS. The ordinate represents the survival rate, and the abscissa the time passed (in days).

FIG. 4 is a graph showing protection against tumor challenge with ST40L. It shows the relationship between the number of A20 cells that were grafted as the tumor challenge and the survival rate. The ordinate represents the survival rate, and the abscissa the time passed (in days).

FIG. 5 a graph demonstrating protection against tumor challenge with ST40L, comparing the temporal relationship between the challenge by A20 cells and the inoculation of ST40L. (The inoculation was done prior to the challenge.) The ordinate represents the survival rate, and the abscissa the time passed (in days).

FIG. 6 is a graph showing protection against tumor challenge with ST40L, comparing the temporal relationship between the challenge by A20 cells and the inoculation of ST40L. (The inoculation was done after the challenge.) The ordinate represents the survival rate, and the abscissa the time passed (in days).

FIG. 7 shows an immunostaining image, with an anti-Fas ligand, of a tumor tissue from the mouse treated with PBS to detect Fas ligand expression. A region where lymphocytes expressing the Fas ligand infiltrate is indicated by an arrow.

FIG. 8 shows an immunostaining image, with an anti-Fas ligand, of a tumor tissue from the mouse treated with ST, to detect the Fas ligand expression. Regions where lymphocytes expressing the Fas ligand infiltrate are indicated by arrows.

### DETAILED DESCRIPTION OF THE INVENTION

The term “vaccine” used herein means a composition that induces immune reactions in the host. “Tumor immunity” means the host's immune response to a tumor. An “exogenous gene” means a gene that is artificially introduced from outside the cell. Therefore, even if the gene is derived from the same species as the host, as far as the gene is artificially introduced to the cell, it is an exogenous gene. A “microbial cell that can transfer an exogenous gene into the host cell” means the cell of a microorganism that can transfer an exogenous gene of interest into the host cell upon inoculation through a certain administration route. It is desirable that the microbial cell is nonpathogenic or sparingly pathogenic to the host at least when introduced by the administration method utilized. In addition, a “DNA that can induce tumor immunity in the host” means a DNA whose expression induces immune response to the tumor.

DNAs that can induce tumor immunity used in the present invention generate, by their expression, RNAs or proteins that augment tumor immunity. A representative example of such DNA is the DNA encoding CD40L. As described earlier, CD40L stimulates the host's immune system, and at the same time, increases the immunogenicity of B lymphoma cells, thereby augmenting the tumor immunity.

In addition, DNAs encoding tumor-specific antigens such as carcinoembryonic antigen (CEA) can also be DNAs that can induce tumor immunity since their expression products function as cancer vaccines. Dranoff et al. introduced various cytokine genes into malignant melanoma cells ex vivo and administered the cells into mice as vaccines, detecting a very strong tumor immunity-augmenting effect when the granulocyte-macrophage colony stimulating factor (GM-CSF) gene was used. In fact this treatment for malignant melanomas using the GM-CSF gene has been producing fruit (G. Dranoff et al., Proc. Natl. Acad. Sci. USA 90: 3539-3543, 1993; G. Dranoff et al., Hum. Gene Ther. 8: 111-123, 1997; S. P. Leong et al., J. Immunother. 22: 66-74, 1999; and R. Soiffer et al., Proc. Natl. Acad. Sci. USA 95: 13141-13146, 1998). The DNA encoding GM-CSF can also be utilized as the DNA that augments tumor immunity in the present invention.

Examples of the DNAs used in the present invention that can induce tumor immunity include those encoding the polypeptides described below. These polypeptides have been identified as antigens recognized by T cells of tumors that are also described below (Saibo Kogaku 17(8), 1194-1199, 1998).

| Tumor antigens | Corresponding tumors |
|---|---|
| Tissue-specific protein | |
| melanosome protein (gp100, | melanoma |
| MART-1/Melan-A, TRP*1, TRP2, | |
| tyrosynase) | |
| CEA* | colon cancer |
| PSA* | prostate cancer |
| Tumor-specific mutant peptide | |
| β-catenin, CDK*4, MUM*-1 | melanoma |
| CASP*-8 | squamous cell carcinoma |
| Cancer-testis antigen | |
| MAGE*-1, MAGE-3, BAGE, | melanoma, various epithelial |
| GAGE-1, GAGE-2, NY-ESO-1 | cancers |
| Cancer gene product | |
| HER2/neu | mammary cancer, ovarian cancer, |
| | lung cancer |
| p53 | squamous cell carcinoma |
| Ras | colon cancer, thyroid cancer |
| Viral proteins | |
| HPV*16 protein (E7) | cervical cancer |
| EBV* protein (EBNA*-2, EBNA-3, | B cell lymphoma |
| EBNA-4, EBNA-6, LMP*2) | |
| Idiotype | |
| antibody (variable region) | B cell lymphoma |
| Mutant HLA | |
| HLA-A2 mutant | stomach cancer |
| Others | |
| p15, GnT-V* | melanoma |
| PRAME* | melanoma, various cancers, |
| | leukemia |
| SART-1* | esophagus cancer, cephalic and |
| | cervical squamous cell |
| | carcinoma, lung squamous cell |
| | carcinoma, adenocarcinoma |
| F4, 2 | stomach cancer |
| MUC 1 | mammary cancer, ovarian cancer, |
| | pancreatic cancer |
| RAGE*-1 | stomach cancer |
| *Note: each abbreviation represents as follows: CASP, Caspase; CDK, cyclin-dependent kinase; CEA, carcinoembryonic antigen; EBNA, EBV-determined nuclear antigen; EBV, Epstein-Barrvirus GnT-V, N-acetylglucosamyltransferase-V; HPV, human Papillomavirus; LMP, latent membrane protein; MAGE, melanoma antigen; MUM, melanoma ubiquitous mutated; PRAME, preferentially expressed antigen of melanoma; PSA, prostate-Specific antigen; RAGE, renal tumor antigen; SART-1, squamous cell carcinoma antigen recognized | |
| # by T cells-1; TRP, tyrosinase-related protein. | |

In the present invention the origin of CD40L is not particularly restricted, however, it is preferably derived from the same species as the host to be treated. This is because even though CD40L from another species can be expected to generate a similar effect, one cannot exclude the possibility of this CD40L being eliminated by the host's immune system in the long run. CD40L used in the present invention does not act as an antigen in the host, but is expected for its immune-stimulating activity. Therefore, an immune response of the host to CD40L is an undesirable side effect. On the other hand, CD40L is not necessarily derived from the same species when an action as an immunogen like CEA to eliminate CEA is expected as long as it induces immune response against CEA.

In the present invention, the DNA that induces tumor immunity can be chosen depending on the type of cancer to be treated. For example, if one uses the DNA encoding CD40L, one can expect its effect especially against CD40L positive lymphomas. In contrast, if one uses the DNA encoding tumor antigens such as CEA, which is specifically expressed in colon cancer, one can expect its effect against tumors expressing that particular antigen.

Since CD40L stimulates the immune system together with other cytokines, the antitumor activity is expected to be augmented by co-transfection with interleukin 10, 12, various interferons, GM-CSF, etc. Co-transfection can be performed by, for example, introducing a cytokine gene into the Salmonella bacterium by the same method as used to introduce the CD40L gene into the Salmonella bacterium, and administering both simultaneously. Alternatively, another antibiotic resistance gene, such as a chloramphenicol resistance gene, is introduced into the cytokine expression vector, and the Salmonella bacterium that has both CD40L and cytokine genes can be selectively amplified in the presence of the two kinds of antibiotics. It is also possible to express multiple genes at the same time by cloning genes in multiple vectors or under multiple promoters. It is also possible to combine a single promoter and the Internal Ribosomal Entry Site (IRES) sequences.

CD40L is a type II membrane protein with a molecular weight of approximately 39 kD, which belongs to the TNF family (Armitage, R. J. et al., Nature 357: 80-82, 1992). It is composed of an extracellular domain of 215 amino acids and an intracellular domain of 22 amino acids on the cell membrane. The DNA encoding the human CD40L has already been cloned (ATTC Number: 79814, http://www.atcc.org/; FEBS Lett. 315: 259-266, 1993), and its nucleotide sequence (GenBank Accession No. Z15017) is publicly available (EMBO J. 11: 4313-4321, 1992). When one uses CD40L in the present invention, it is not always necessary to use DNA encoding the entire domains. Namely, one may use a DNA fragment that expresses only the activity domain of CD40L. For example, CD40L exists not only on the cell surface but also in the blood (the soluble form), and the latter is known to have a similar activity to the cellular form (Lane P., J. Exp. Med. 177: 1209-1213, 1993). It is also estimated that the receptor binding domain of CD40L is composed of the C-terminal 150 amino acids in the extracellular domain.

DNAS that can induce tumor immunity used in the present invention are retained in microbial cells in such a state as to be expressed in the host cell. The “state so as to be expressed in the host cell” means, for example, a state in which the DNA is operably linked to a promoter that functions in the host cell. More concretely, for example, if the host is a mammal such as a human, the DNA is ligated to a promoter such as the CMV promoter which controls expression of the DNA to allow its expression in the host cell.

In this case, a Tet on/off system such as the T-RE_{X} system (commercial name) of Clontech enables artificially controlling the expression of the gene through the administration of tetracycline, even after the gene of interest has been introduced into the body. The T-RE_{X} system controls the exogenous gene expression through the administration of tetracycline by combining an expression vector pcDNA4/TO with a control vector pcDNA6/TR. Namely, the Tet repressor (TetR), which is the expression product of the control vector, binds to the Tet operator (TetO₂) on the expression vector to repress the expression, however, tetracycline inhibits the binding, thereby removing the repression of expression.

A promoter functioning only in bacterial cells can be used in the control vector used in the Tet on/off system. This system needs only the TetR supply in the principle. In this method, TetR exists in the bacterial cell as a plasmid, which binds to Teto₂. Administering tetracycline to this cell relieves the expression repression by TetR. The TetR supply does not continue in the eukaryotic cell because the control vector does not function in this cell. Therefore, the removal of the expression repression occurs irreversibly, and the single administration of tetracycline can initiate the expression of the expression vector. Namely, one does not need to maintain constant concentrations of tetracycline and uses it only as a trigger.

For the expression in mammalian cells, the DNA to be expressed is inserted into a known mammalian expression vector and a microbial cell is transformed with the vector. Suitable mammalian expression vectors include retrovirus-derived expression vectors, such as pLNCX (Clontech), pSI (Promega), which can exist in a high copy number of plasmid vector in the bacterial cells, and pCI (Promega) having a human cytomegalovirus immediately-early enhancer/promoter region which enables strong and stable expression in the mammalian cell, etc. Expression of a desired protein by transfection of the expression vector is not always performed in an optimal state. The double-stranded RNA generated through the transfection activates dsRNA-activated inhibitor (DAI), which is one of the anti-viral defense system enzymes in the target cell, the activated DAI phosphorylates the translation initiation factor eIF-2, and it terminates the translation. This inhibition of translation by DAI can be overcome by the adenovirus Virus Associated I RNA (VAI RNA). For example, the pAdVAntage Vector (Promega) containing this VAI RNA gene is co-transfected with the expression vector for the desired protei to elevate the expression level of the desired protein. On the other hand, the PCR products prepared with heat-resistant enzymes such as Taq DNA polymerase have a single base overhang of adenine on the DNA terminus. Thus, a gene encoding the desired protein amplified by PCR can be directly ligated with a mammalian expression vector with a thymidine single base overhang on its DNA terminus, such as pTARGET Vector (Promega) and pCR3.1 (Invitrogen).

The vector, into which the DNA to be expressed in the host has been inserted, is transfected into a microbial cell that introduces exogenous genes into the host cell. The exogenous genes can be introduced into the host cell usually by intrusion of the microbial cell into the host. The microbial cell intrudes into the cytoplasm of the host cell and releases the retained vector into the host cell, thereby introducing the exogenous genes.

An example of the microbial cell capable of introducing exogenous genes into mammalian cells is attenuated Salmonella bacterium. The “attenuated Salmonella bacterium” used in the present invention means Salmonella bacterium whose pathogenicity to the host is attenuated while it is still capable of penetrating the intestinal epithelium and introducing the exogenous genes into the lymphatic system. The pathogenicity of the Salmonella bacterium to the host can be attenuated by, for example, deleting its inherent ability to proliferate within the cell by means of mutation. Known attenuated Salmonella bacterium is exemplified by the auxotrophic aroA⁻ strain of Salmonella typhimurium, which is the causative microbial agent of typhoid fever. Such mutants can be constructed by the method described in S. K. Hoiseth and B. A. Stocker, Nature 291: 238-239, 1981. It should be noted that the attenuated Salmonella bacterium used in the present invention not only functions as a carrier of genes but also augments the tumor immunity-inducing effect. The attenuated Salmonella bacterium used in Examples (S. typhimurium aroA⁻ strain) is thought to promote the secretion of IFN-γ and strongly stimulate Th1. The action of CD40L is strongly influenced by cytokines. For example, it highly promotes the production of IgE in the presence of IL-4. Therefore, the use of the attenuated Salmonella bacterium can produce a remarkable tumor immunity-inducing effect that cannot be achieved by only the expression of DNA that induces tumor immunity. Thus, the attenuated Salmonella bacterium used in the present invention functions as a potentiator for the tumor immunity induction activity of CD40L.

The attenuated Salmonella bacterium, through oral inoculation, infects the digestive tract of the host, and intrudes into the cytoplasm. The attenuated Salmonella bacterium must be a live bacterium because it needs to penetrate the intestinal epithelium. The intruded bacterial cells divide several times in the cytoplasm, then cease proliferation to die out. During this process, the vector retaining exogenous genes is introduced into the host cell. As described in Examples, the vector containing the DNA to be expressed reaches the Peyer's patch through the small intestine and expresses the inserted DNA. Since the Peyer's patch is a tissue that functions as the interface between the digestive tract tissues and the lymphatic system, CD40L can directly act on the immune tissues.

Microbial cells can be transformed with a vector into which an exogenous gene has been inserted by known methods such as electroporation. For example, the pcDL-SR vector can be introduced into the attenuated Salmonella bacterium by adding the vector to the microbial cells suspended in 10% glycerol, applying a single pulse of 12.5 kV/cm (2.5 kV, 200Ω, 2.5 μF) in a cuvette chilled on ice, and immediately adding 1 ml of pre-warmed SOC. The transformant recovered by using the drug resistance marker inherent to the vector as an indicator can be used as a cancer vaccine of the present invention.

A transformant can be used as a vaccine as it is or by mixing with physiologically acceptable carriers. Physiologically acceptable carriers include physiological saline, vegetable oils, suspending agents, surfactants, and stabilizers. Preservatives and other additives can also be added. It is also possible to add immunopotentiators such as cytokines, cholera toxin, and salmonella toxin to enhance immunogenicity. The vaccine preparations of the present invention can be formulated into suspensions or dried powders, or into any other dosage forms by capsulation or compression shaping. If the attenuated Salmonella bacterium is used as the gene carrier, its inoculation route can be oral administration. Thus, it can be added to any food to serve as the food for cancer therapy.

Although the dose of the vaccine of the present invention can vary depending on the dosage form of vaccine and the administration method, a person skilled in the art can choose an appropriate dose as necessary. When the attenuated Salmonella bacterium is used as the gene carrier to express CD40L, it can produce more effective immune activity based CD40L's mechanism of action if the CD40L is efficiently supplied to the immune system of the host. CD40L induces tumor immunity by strengthening the host's immune functions. Therefore, the system of the present invention introduces the vector into M cells of the Peyer's patch by utilizing the attenuated Salmonella bacterium to express CD40L, thereby very effectively inducing tumor immunity. This is also clear from the results shown in the Examples. If the vector containing the DNA to be expressed is administered directly into, the spleen, the lymph nodes, and the like, a desired gene could not be efficiently introduced. Thus, the oral administration using the attenuated Salmonella bacterium as the gene carrier can be an extremely desirable embodiment of the present invention.

The cancer vaccines of the present invention are used for preventing and treating cancer. Namely, the vaccines of the present invention can be used prophylactically for a patient who may possibly develop a cancer. For example, it is possible to administer a vaccine of the present invention to a patient who has completed the treatment of cancer by such therapies as chemotherapy or irradiation therapy to prevent recurrence. Cancers of the hematopoietic system such as leukemia and malignant lymphoma are known to be relatively easily remitted. Once these cancers recur, they become resistant to the treatment conducted so far, which makes it difficult to treat these recurred cancers. Therefore, most desirable use of the cancer vaccines of the present invention is for preventing recurrence of these cancers. Especially, the combined use of the DNA encoding CD40L and the attenuated Salmonella bacterium would produce a strong tumor immunity-inducing activity of CD40L against the B-cell type tumors. In addition, the vaccines of present invention can be used for preventing not only recurrence of these cancers but also onset of other cancers.

For therapeutic purposes, the vaccines of the present invention are orally administered to patients suspected of cancer. In this case, a higher therapeutic effect can be expected if combined with chemotherapy or irradiation therapy. In general, the smaller the. tumor size is, the higher the obtained therapeutic effect is. Thus, combinations of various anticancer therapies can suppress the tumor growth. The vaccines of the present invention can be applied to both the B-cell type tumors and the tumors of the hematopoietic system. In addition, a wide variety of cancers can be targeted by choosing appropriate DNA that can induce effective tumor immunity depending on the tumor to be prevented or treated.

The tumor immunity-inducing effect can be maintained for a long period of time of by administering the vaccines of the present invention about every two month.

The present invention provides cancer vaccines that can effectively induce tumor immunity in the host. Namely, the present invention enables inducing a high level tumor immunity, which are impossible to achieve by the known methods, by specifically augmenting the host's tumor immunity through the expression of DNA capable of inducing tumor immunity, and using a microbial cell that augments tumor immunity as the gene carrier. Especially, the combination use of a vector capable of expressing CD40L and the attenuated Salmonella bacterium as the gene carrier enables the expression of CD40L in the lymphatic system of mammals by such a simple method as oral administration. The CD40L expressed in the lymphatic system can directly act on the host's immune system, and effectively induce tumor immunity. In this combination, CD40L works effectively against B-cell type tumors for which the prevention of recurrence should be sought.

The present invention is demonstrated with reference to the following Examples, but is not to be construed as being limited thereto.

### EXAMPLE 1

### Introduction of CD40L Gene into Mice Using Salmonella

The auxotrophic ST aroA⁻ strain SL5000 (Hoeseth S. K. et al., Nature 291: 238, 1981) was used as a gene carrier. The full length human CD40L gene was cloned into pcDL-SR (ATCC Number: 79814; pcDL-SralphahCD40L Or) using a CMV promoter and used as expression vector for CD40L. The Salmonella strain was transformed with the vector by a conventional method (High efficiency transformation of Salmonella typhimurium and Salmonella typhi by electroporation. Mol. Gen. Genet. 223: 156-158, 1990). The auxotrophic ST aroa⁻ strain SL5000 was grown in 100 ml L broth (Sigma Diagnostics, St. Louis, Mo.) to A₆₀₀ of 0.6, chilled on ice, and harvested by centrifugation (15 min, 1000×g at 4° C.). The pellet was suspended in a final volume of 200 μl in 10% glycerol. Aliquots (40 μl) were mixed with 1 to 2 μl DNA in a chilled microcentrifuge tube and transferred to chilled cuvettes (0.2 cm electrode gap). A single pulse of 12.5 kV/cm (2.5 kV, 200Ω, 25 μF) was applied and 1 ml of prewarmed SOC was immediately added. The bacteria were transferred to 17×100 mm polypropylene tubes and shaken for 1 hour at 37° C. before being plated onto LB agar with ABPC (50 ng/ml).

Groups of 6-8 female BALB/c mice were fed with 0.5 ml phosphate buffered saline (PBS) containing 10⁹ colony-forming units (CFU) of ST with or without CD40L gene. None of the mice exhibited any overt signs of illness during immunization.

### EXAMPLE 2

### Expressions of Fas, B7-1, and B7-2 in NIH3T3 Cells into which Human CD40L Gene is Introduced

A20 is a BCL cell line derived from BALB/c mice and expressing CD40 as well as the major histocompatibility complex (MHC) class I and class II h-2d, IgG, and Fc receptor (ATCC Number: TIB-208). We first examined the expression of Fas, B7-1, and B7-2 on A20 cells cultured with NIH3T3/vt or NIH3T3/CD40LT cells fixed in formalin by immunofluorescence flowcytometry. NIH3T3 cells transfected with human CD40L gene in pcDL-SR expression vector (NIH3T3/CD40LT) and NIH3T3 cells transfected with vector alone (NIH3T3/vt) were prepared by electroporation. Transfectants were selected by growth in 400 μg/ml G418 and then subcloned. The cells were harvested at 70% confluence, washed three times in PBS (Sigma) to remove G418, and fixed in 1% formalin (Sigma) for 10 min at room temperature. After six further washed with PBS, the cells were cultured with A20 cells (100 A20 cells/NIH3T3 cell) for 48 hours in 10% FBS+RPMI1640.

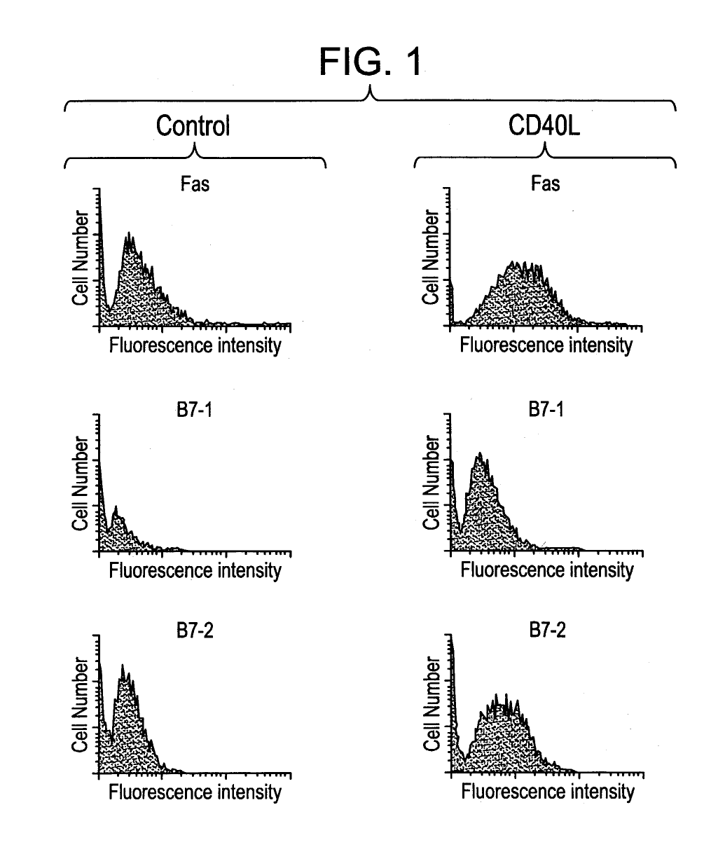

Phenotypic changes of A20cells (1×10⁶/ml) treated with NIH3T3/D40LT (1×10⁴/ml) or NIH3T3/vt (1×10⁴/ml) for 48 hours were examined using flowcytometric analysis. Antibody-coated cells were enumerated by flowcytometric analysis using an EPICS V cell sorter (Coulter Electronics, Hialeah, Fla.). The following antibodies (Abs) were used: FITC-conjugated hamster anti-mouse CD80 (B7-1) Ab (hamster IgG) (Pharmigen, SanDiego, Calif.), FITC-conjugated rat anti-mouse CD86 (B7-2) Ab (IgG2a,k) (Pharmigen), and FITC-conjugated hamster anti-mouse CD95 (Fas) Ab (hamster IgG) (Pharmigen). The expression levels of Fas and B7-2 on the surface of A20 cells cultured with NIH3T3/vt (control) were found to be low or undetectable, while NIH3T3/CD40LT cells upregulated the expression of Fas, B7-1, and B7-2 molecules (FIG. 1).

### EXAMPLE 3

### Expression of Human CD40L Protein Invarious Murine Tissues

In order to analyze expression of the human CD40L protein in murine tissues, samples from the small intestine, colon, liver, and spleen, were analyzed by hematoxylin-eosin (HE) staining as well as immunohistochemistry. Paraffin embedded specimens were used for HE staining. For immunohistochemical staining, frozen tissue sections were treated with anti-human CD40L antibody (Santa Cruz).

HE staining was performed as follows. First, slides of paraffin embedded specimens were prepared and treated with xylene for 10 to 15 minutes twice. Deparaffinization was performed by washing the slides with 100% alcohol for 5 to 10 minutes, with 90% alcohol for 5 to 10 minutes, with 80% alcohol for 5-to 10 minutes, with 70% alcohol for 10 to 15 minutes. In this way, the water content of washings was gradually increased, and finaly water was used. The slides were then washed again with running water, soaked in hematoxylin solution for 0.5 to 15 minutes, and rinsed with running water for 20 to 30 minutes. Staining intensity was adjusted, if necessary, by warming the staining system with tepid water. If the staining was too strong, a hydrochloric acid and alcohol mixture (100 ml of 70% alcohol+concentrated hydrochloric acid) was added. Next, the slides were soaked in eosin solution, which was then gradually substituted with organic solvent, i.e. 70% alcohol for about 5 minutes, 80% alcohol for about 5 minutes, 90% alcohol for about 5 minutes, 95% alcohol for about 5 minutes, and finally 100% alcohol for about 5 minutes, monitoring the intensity of each staining under a microscope. The slides were then soaked in xylene for 10 to 15 minutes twice and embedded into paraffin.

We found that in mice immunized with ST40L, the Peyer's patches were prominent, and the majority of cells in the Peyer's patches could be seen to express the human CD40L protein. There were a few CD40L⁺ cells in spleen, but not in liver. In contrast, human CD40L was not detectable in the Peyer's patches of mice treated with ST or PBS.

### EXAMPLE 4

### Confirmation of Secretion of Human Soluble CD40L by Transfected Murine Cells into Sera

To further confirm the secretion of human soluble CD40L by transfected murine cells into the sera, we next examined it by ELISA.

Soluble human CD40L levels in the sera of BALB/c mice were quantified using the soluble CD40L enzyme-linked immunosorbent assay (ELISA) kit (Chemicon International, Inc.), which utilized a sandwich based immunoassay design. The minimal detection level was 0.16 ng/ml of soluble CD40L.

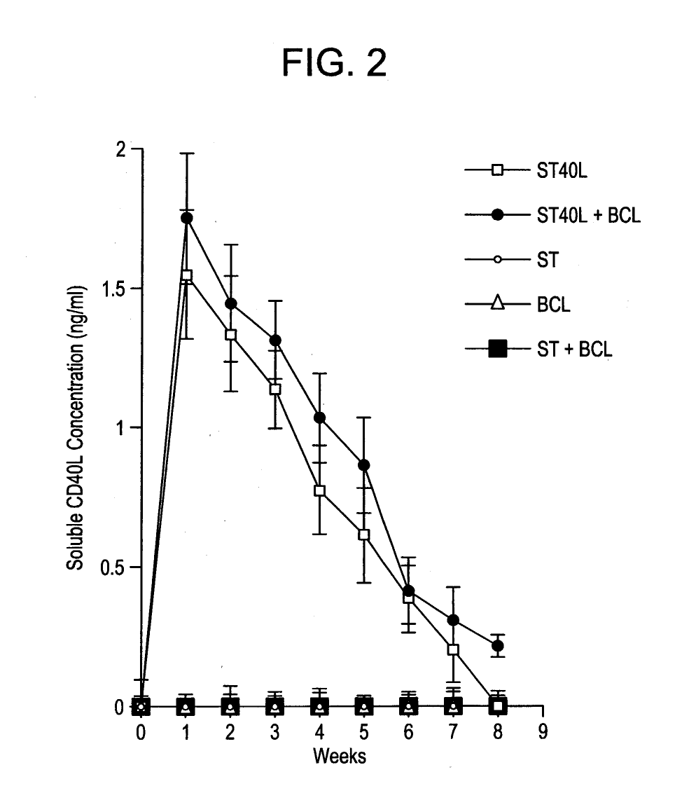

Human soluble CD40L protein was detectable only in BALB/c mice treated with ST40L with or without administration of BCL cells, but not detectable in mice treated with ST and/or BCL cells (FIG. 2).

### EXAMPLE 5

### Effects of Vaccines of the Present Invention Orally Administered on the Survival Rate of Mice Inoculated with BCL Cells

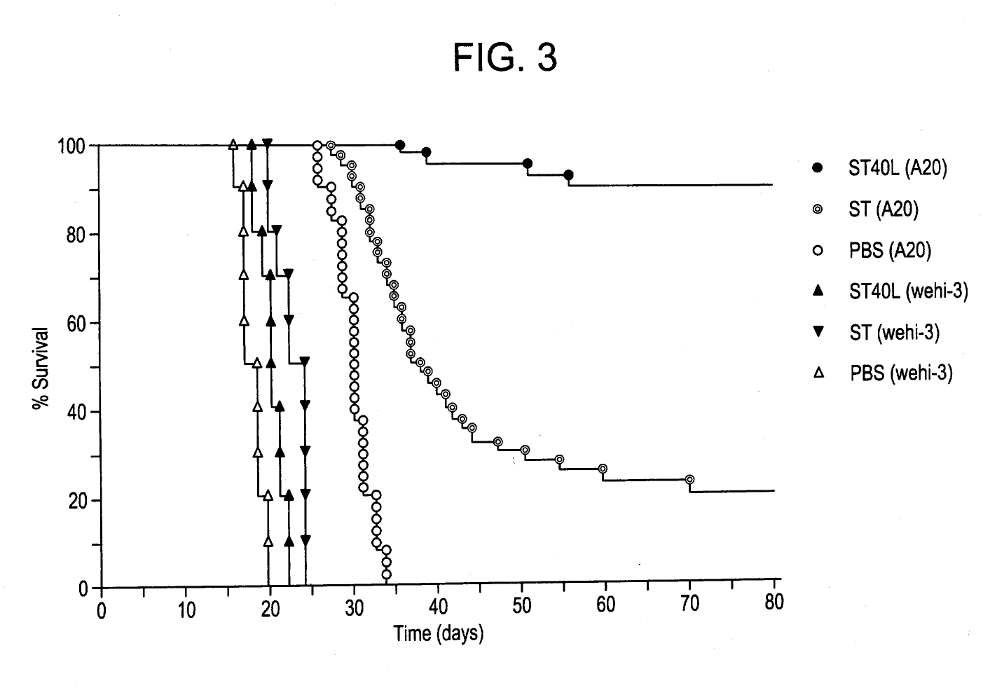

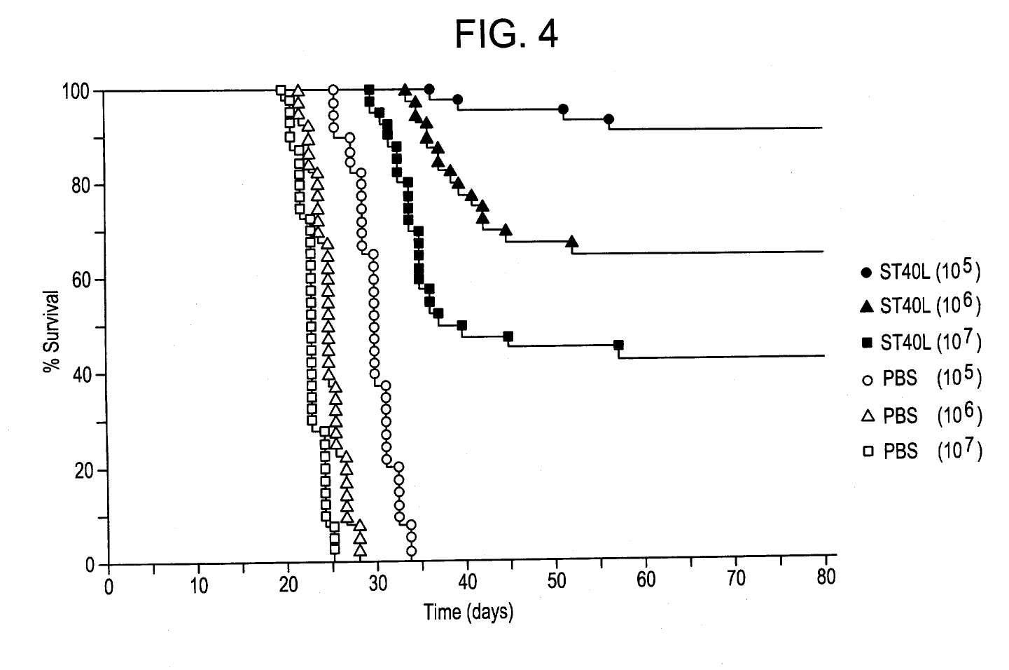

BALB/c mice were injected subcutaneously (SC) with 10⁵ A20 cells, these mice were then orally administrated with ST40L, ST, or PBS alone, and their survival monitored. Mice in the group treated with ST40L were found to have a significantly longer survival than those treated with ST or PBS (Kaplan-Meier method: Mantel-Cox, p<0.0001) (FIG. 3). The mice in the group treated with ST alone also survived for a significantly longer period than those treated with PBS alone (p<0.0001). In contrast, SC injection of an equivalent number of CD40negative wehi3 leukemia cells showed that there was no significant difference between treatments with ST40L, ST, and PBS. When differing numbers of A20 cells (10⁵, 10⁶, or 10⁷) were injected, the survival rates of the mice were 92%, 77%, and 55%, respectively (FIG. 4).

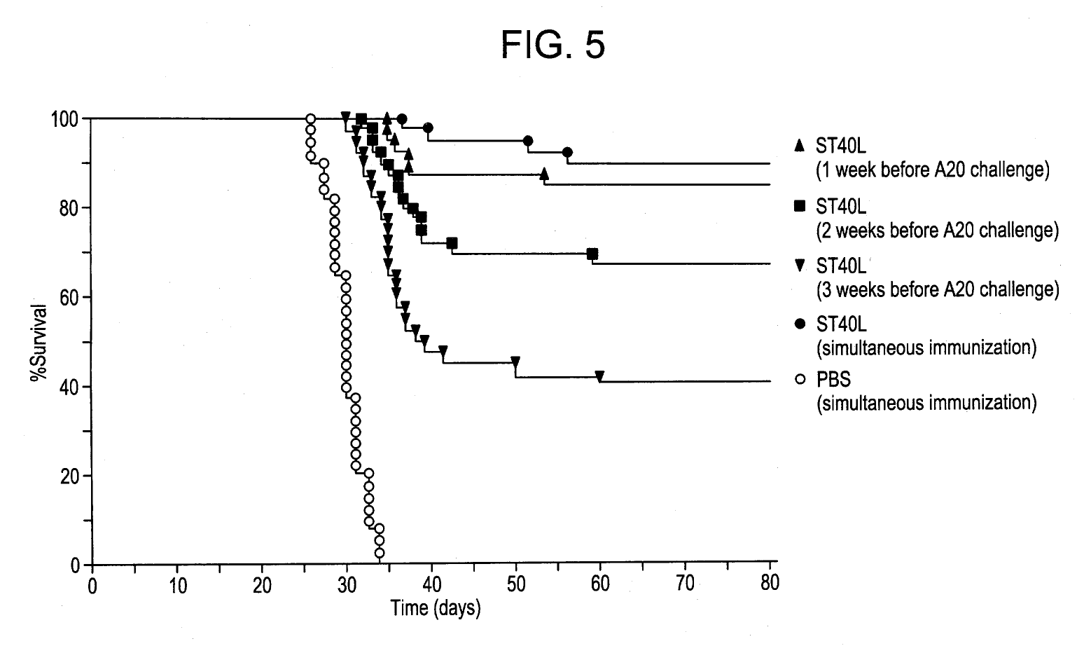

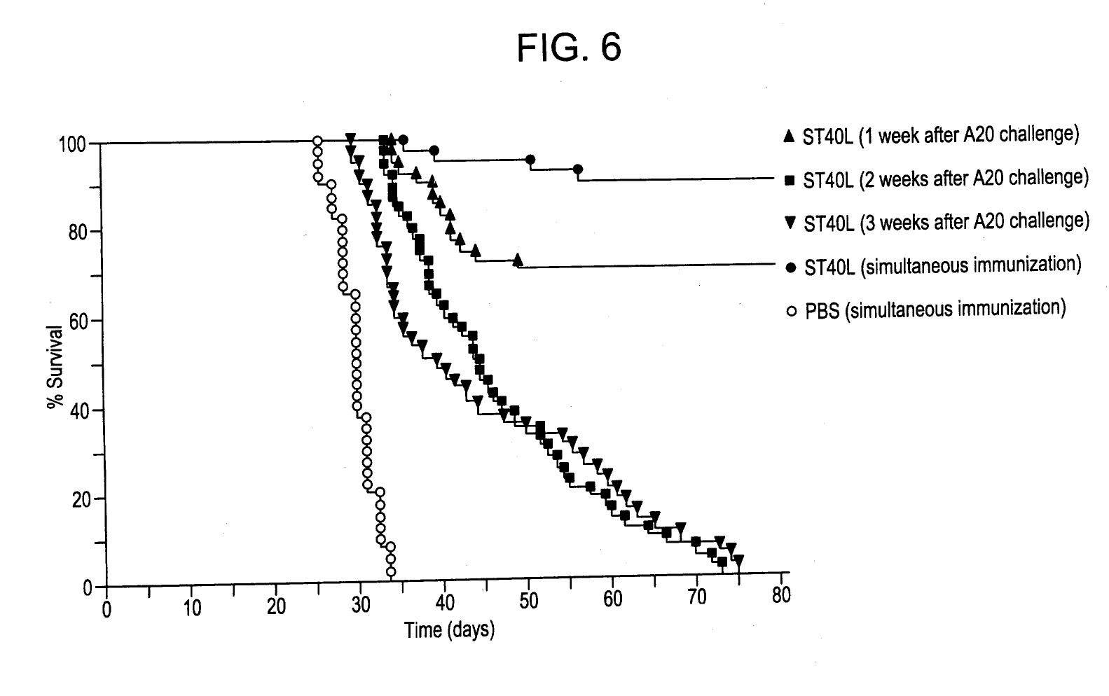

When ST40L was administrated one week prior to tumor cell challenge (10⁵ A20 cells SC injection), no significant differences were observed in mice survival compared with simultaneous vaccination by SC injection of A20 cells alone. However, the efficiency of the ST40L was found to be decreased when the mice were immunized at either three weeks (52%, p<0.01), or two weeks (67%, p<0.05); prior to A20 cell (10⁵) SC injection (FIG. 5). This effect was also seen when the mice were ST40L immunized at three weeks (42%, p<0.01), two weeks (69%, p<0.001), or one week after (70%, p<0.02) A20 cell (10⁵) SC injection (FIG. 6).

### EXAMPLE 6

### Histological Analysis of Tumor Tissues from Mice Treated with ST40L

To explore the mechanisms of the protection from BCL growth, Fas ligand expression was examined in tumor tissue from mice treated with ST40L, ST, or PBS alone. Samples of tissues excised from paraffin sections were reacted with an anti-Fas ligand antibody and further reacted with peroxidase-conjugated goat anti-rabbit antibody as the second antibody. Substrates were then added.

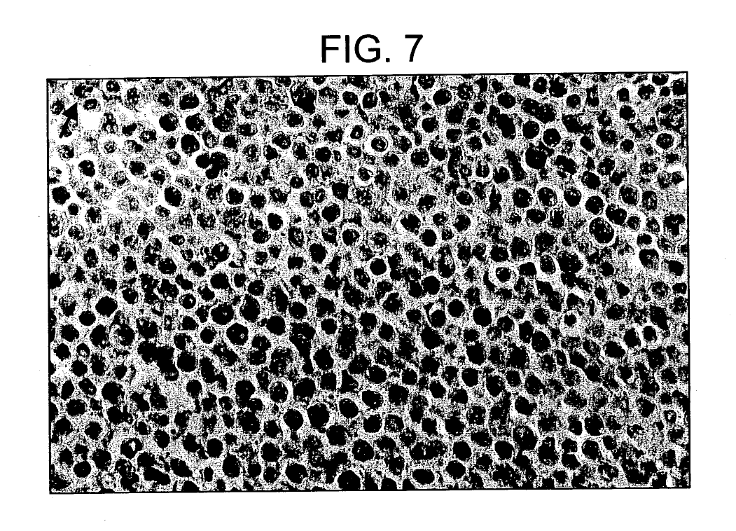

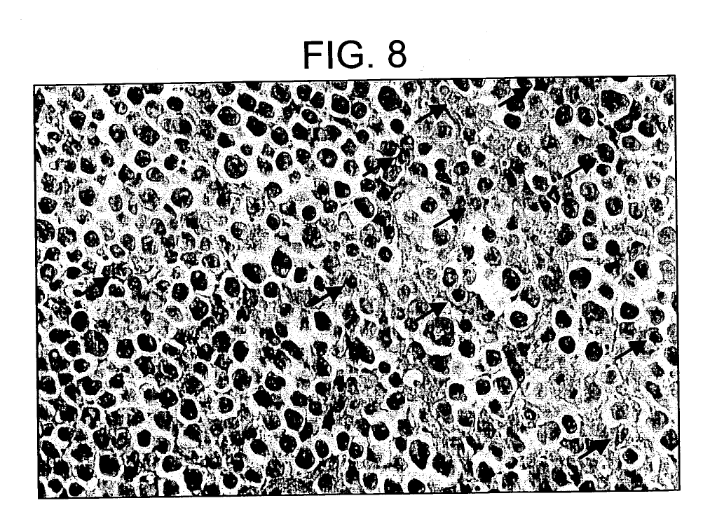

In the mice treated with PBS alone, no cellular infiltrate expressing Fas ligand was observed in the surrounding tissues and inside the BCL region (FIG. 7). In contrast, infiltrating lymphocytes expressing Fas liqand were observed around the vessels and also scattered in the smaller tumor tissues in the mice treated with ST (FIG. 8). Small hard nodules (2 to 5 mm in diameter) were observed at the SC injection-sites of the long-term survival mice that had been treated with ST40L. On histological analysis, these small nodules were confirmed to be the result of an accumulation of lymphocytes, and not BCL cells, and these lymphocytes were also found to be strongly positive for Fas ligand expression.

What is claimed is:

1. A cancer vaccine comprising a microbial cell that includes an exogenous DNA that induces, in a host organism, tumor immunity.

2. The cancer vaccine of claim 1, wherein the microbial cell is an attenuated Salmonella bacterium.

3. The cancer vaccine of claim 2, wherein the attenuated Salmonella bacterium is an auxotrophic aroA⁻ strain of Salmonella typhimurium.

4. The cancer vaccine of claim 2 or 3, wherein the vaccine is a preparation for oral administration.

5. The cancer vaccine of claim 1, wherein the DNA encodes a CD40 ligand.

6. The cancer vaccine of claim 1, wherein said microbial cell transfers said exogenous DNA into a cell of said host organism.

7. The cancer vaccine of claim 1, wherein said microbial cell absent transfer of said exogenous DNA to said host cell induces tumor immunity in said host organism.
